(19) United States
(12) Patent Application Publication
(10) Pub. No.: US 2026/0114790 A1
(43) Pub. Date: Apr. 30, 2026

(54) BALANCE SYSTEM DEVELOPMENT TRACKING

(71) Applicant: Cochlear Limited, Macquarie University, NSW (AU)

(72) Inventor: Christopher Joseph LONG, Centennial, CO (US)

(21) Appl. No.: 18/835,592

(22) PCT Filed: Feb. 2, 2023
(86) PCT No.: PCT/IB2023/050917

§ 371 Date: Aug. 2, 2024

Related U.S. Application Data
(60) Provisional application No. 63/307,341, filed on Feb. 7, 2022.

(51) Int. Cl. A61B 5/00; A61B 5/11; A61B 5/12

(52) U.S. Cl. CPC A61B 5/4023; A61B 5/1123; A61B 5/123; A61B 5/6803; A61B 5/7246; A61B 2503/04

(57) ABSTRACT

Presented herein are techniques for identifying pediatric milestones using a medical device, including implantable medical devices and hearing devices, based upon motion data and acoustic data obtained by the medical device. For example, when the medical device is embodied as a hearing device, such as a cochlear implant or hearing aid, a pediatric milestone of a user of the hearing device may be determined based on motion data and acoustic data associated with the user.

### BACKGROUND

### Field of the Invention

The present invention relates generally to detecting and tracking pediatric developmental milestones.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: obtaining motion data from at least one motion sensor in a hearing device configured to be worn on a head of a user; obtaining acoustic data from at least one acoustic detector in the hearing device; and determining, based on the motion data and the acoustic data, whether the user is meeting one or more predetermined developmental milestones.

In another aspect, one or more non-transitory computer readable storage media are provided. The one or more non-transitory computer readable storage media comprise instructions that, when executed by a processor, cause the processor to: obtain motion data from at least one motion sensor in a hearing device configured to worn on a head of a user; obtain acoustic data from at least one acoustic detector in the hearing device; and determine, based on the motion data and the acoustic data, whether the user is meeting one or more predetermined developmental milestones.

In another aspect, a hearing device is provided. The hearing device comprises: one or more motion sensors; one or more acoustic detectors; and one or more processors, wherein the one or more processors are configured to: obtain motion data associated with a user of the hearing device from the one or more motion sensors; obtain acoustic data associated with the user of the hearing device from the one or more acoustic detectors; and determine, based on the motion data and the acoustic data, whether the user of the hearing device is meeting one or more predetermined developmental milestones.

In another aspect, a method is provided. The method comprises detecting motion data associated with a user of a hearing device; determining an amount of delay between a first time when a voice associated with the user is detected and a second time when an echo of the voice is detected; and identifying one or more developmental milestones for balance or motor functions associated with the user based on the motion data and the amount of delay.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a user wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIGS. 2A, 2B, and 2C illustrate a plurality of pediatric milestones and accompanying acoustic data received at a hearing device;

FIG. 3 is a table illustrating pediatric milestones and accompanying motion and acoustic data;

FIG. 4 is a flowchart illustrating a first example process for identifying pediatric milestones based on motion and acoustic data;

FIG. 5 is a flowchart illustrating a second example process for identifying pediatric milestones based on motion and acoustic data;

FIG. 6 is a functional block diagram of an implantable stimulator system with which aspects of the techniques presented herein can be implemented; and

FIG. 7 is a schematic diagram illustrating a vestibular stimulator system with which aspects of the techniques presented herein can be implemented.

### DETAILED DESCRIPTION

Presented herein are techniques for detecting and tracking pediatric developmental milestones using, for example, a wearable or implantable device, based on motion data and acoustic data associated with a user of the wearable or implantable device. For example, when the wearable or implantable device is embodied as a hearing device, such as a cochlear implant or hearing aid, a developmental milestone of a pediatric user of the hearing device may be identified based on motion data associated with the pediatric user. In addition, a microphone or acoustic detector of the hearing device may be used to identify the developmental milestones based on an amount of a delay between detecting a voice of the pediatric user and detecting an echo of the voice of the pediatric user as the voice reverberates off of a floor. Attenuation data and frequency content of the echo of the voice of the pediatric user may additionally be used to identify the developmental milestones and/or distinguish between different developmental milestones.

The techniques presented herein may be beneficial for identifying developmental milestones of pediatric users of hearing devices and tracking whether the pediatric users are on target to reach developmental milestones. Parents and pediatricians often find it useful to track developmental milestones to determine if a child is meeting the age-appropriate targets for height, weight, language, etc. If it is detected that a child is not meeting milestones, additional diagnoses or assistance can be provided. In addition, children with hearing loss can also experience vestibular/balance problems. Being able to communicate to parents/caregivers and clinicians whether or not a hearing-impaired child is meeting her developmental milestones could give peace of mind to the parents/caregivers. Additionally, if a child is not meeting developmental milestones, technological interventions or rehabilitations could potentially be prescribed at an early age.

Merely for ease of description, the techniques presented herein are primarily described with reference to a specific implantable medical device system, namely a cochlear implant system. However, it is to be appreciated that the techniques presented herein may also be partially or fully implemented by other types of implantable medical devices, wearable devices, etc. For example, the techniques presented herein may be implemented by other hearing devices or hearing device systems that include one or more other types of hearing devices, such as hearing aids, middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, consumer electronic devices, wearable devices (e.g., smart watches, etc.), etc. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aid, auditory prosthesis, tinnitus therapy devices, etc.) or a device for use by a person with normal hearing (e.g., consumer devices that provide audio streaming, consumer headphones, earphones and other listening devices).

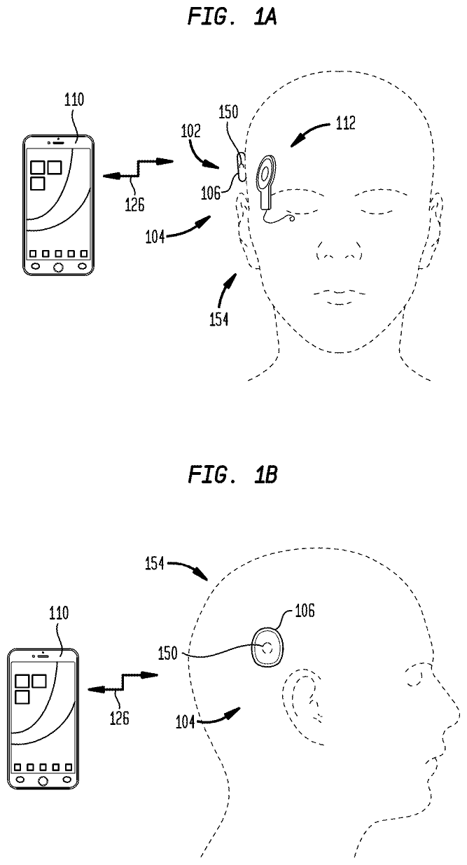

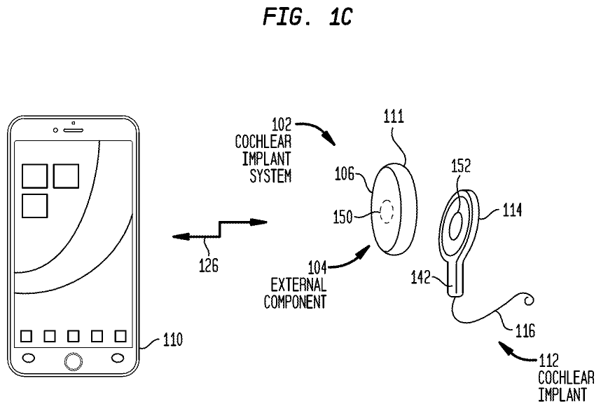

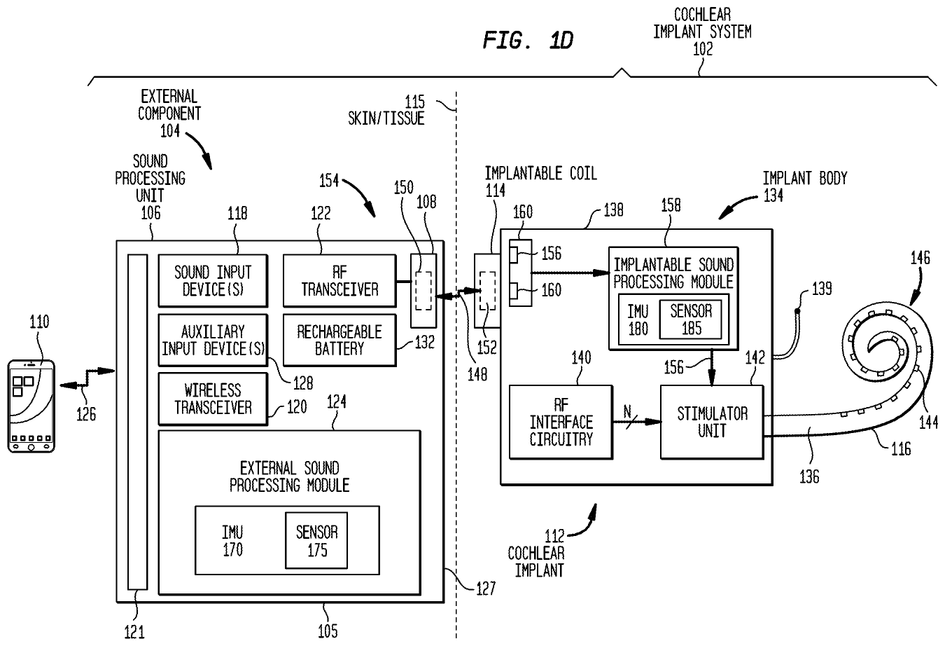

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the user and an implantable component 112 configured to be implanted in the user. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as a radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the user.

According to the techniques of the present disclosure, external sound processing module 124 may include an inertial measurement unit (IMU) 170. The inertial measurement unit 170 is configured to measure the inertia of the user's head, that is, motion of the user's head. As such, inertial measurement unit 170 comprises one or more sensors 175 each configured to sense one or more of rectilinear or rotatory motion in the same or different axes. Examples of sensors 175 that may be used as part of inertial measurement unit 170 include accelerometers, gyroscopes, inclinometers, compasses, and the like. Such sensors may be implemented in, for example, micro electromechanical systems (MEMS) or with other technology suitable for the particular application.

The inertial measurement unit 170 may be disposed in the external sound processing module 124, which forms part of external component 104, which is in turn configured to be directly or indirectly attached to the body of a user. The attachment of the inertial measurement unit 170 to the user has sufficient firmness, rigidity, consistency, durability, etc. to ensure that the accuracy of output from the inertial measurement unit 170 is sufficient for use in the systems and methods described herein. For instance, the looseness of the attachment should not lead to a significant number of instances in which head movement that is consistent with a change in posture (as described below) is not identified as such nor a significant number of instances in which head movement that is inconsistent with a change in posture is not identified as such. In the absence of such an attachment, the inertial measurement unit 170 must accurately reflect the user's head movement using other techniques.

The data collected by the sensors 175 is sometimes referred to herein as head motion data or motion data. As described further below, the head motion data may be utilized to predict milestones of a user of a hearing device.

As also illustrated in FIG. 1D, a second inertial measurement unit 180 including sensors 185 is incorporated into implantable sound processing module 158 of implant body 134. Second inertial measurement unit 180 may serve as an additional or alternative inertial measurement unit to inertial measurement unit 170 of external sound processing module 124. Like sensors 175, sensors 185 may each be configured to sense one or more of rectilinear or rotatory motion in the same or different axes. Examples of sensors 185 that may be used as part of inertial measurement unit 180 include accelerometers, gyroscopes, inclinometers, compasses, and the like. Such sensors may be implemented in, for example, micro electromechanical systems (MEMS) or with other technology suitable for the particular application.

For hearing devices that include an implantable sound processing module, such as implantable sound processing module 158, that includes an IMU, such as IMU 180, the techniques presented herein may be implemented without an external processor. Accordingly, a hearing device that includes an implant body 134 and lacks an external component 104 may be configured to implement the techniques presented herein.

As noted above, implantable medical devices, such as cochlear implant system 102 of FIG. 1D, may include microphones and inertial measurement units, which may provide motion and acoustic data that may be utilized to detect and track one or more aspects of pediatric development. The inertial measurement units may provide data indicating movement data of a user. For example, a rotation around the x-axis may be indicative of rolling over and a forward motion may be indicative of crawling or walking. The microphones may provide data that may contextualize motion data acquired by the inertial measurement units.

In some situations, motion data alone may be insufficient to determine whether a user has reached a developmental milestone. For example, motion data alone may be insufficient to differentiate between crawling and walking since the inertial measurement units may detect similar head motion when a pediatric user is crawling or walking. In such embodiments, an acoustic detector or microphone may capture acoustic data that may be used to determine a location of a user's head with respect to the floor. For example, an acoustic detector may detect reverberations of an acoustic signal, which may indicate a difference between being close to the floor and farther away from the floor. An analysis of the motion data combined with the acoustic data may distinguish crawling from walking.

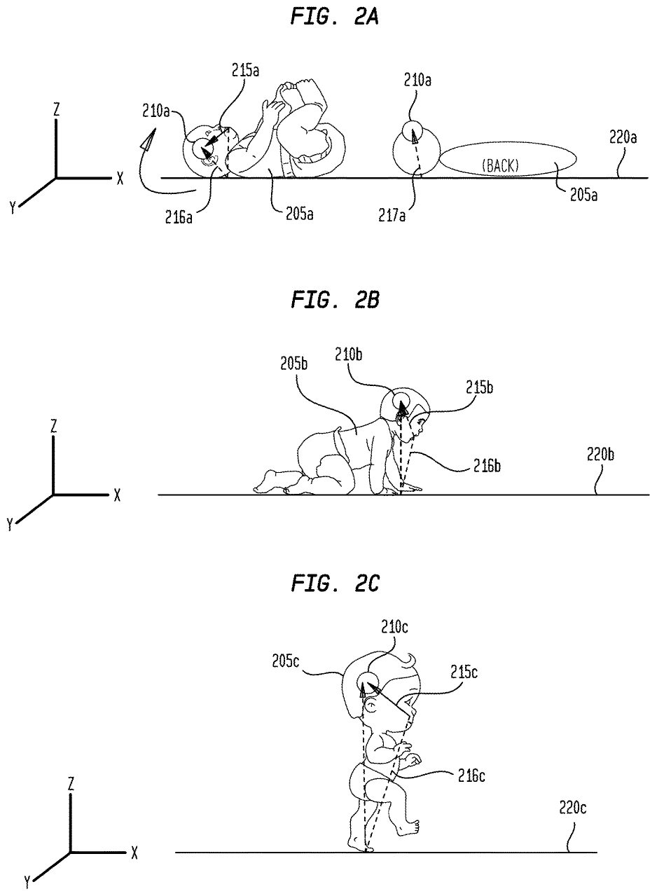

For example, illustrated in FIG. 2A is a pediatric user 205a who begins facing upward toward the ceiling and rolls over in a clockwise direction on floor 220a. While pediatric user 205a is rolling, an inertial measurement unit of hearing device 210a, for example inertial measurement unit 170 or 180 of FIG. 1D, may detect that the head of pediatric user 205a is rotating around the x-axis, indicating a rolling motion. In addition, a microphone or acoustic sensor of hearing device 210 may detect a voice of pediatric user 205 and an echo of the voice of pediatric user 205. For example, the voice of pediatric user 205 may reverberate off of floor 220a and hearing device 210a may detect the voice and the reverberation of the voice.

As illustrated in FIG. 2A, when pediatric user 205a is facing upward toward the ceiling, hearing device 210a is a short distance from the floor 220a. When pediatric user 205a utters a sound, hearing device 210a detects the voice signal, as shown at 215a. In addition, the voice signal travels to floor 220a and hearing device 210a detects an echo of the voice signal, as shown at 216a. Because the hearing device 210a is a short distance from the floor, the delay between hearing device 210a detecting voice signal 215a and detecting the first echo of voice signal 216a is small. The orientation of the head away from the floor means that sound reaching the floor will be low-pass filtered by the head and the sound echoing off the floor will be additionally attenuated and filtered in a way different to the direct sound from the mouth to the hearing device (e.g. the system can consider how the head, torso, and body will filter echoed sounds versus direct sounds from the mouth). As pediatric user 205a continues to roll over and is facing sideways with hearing device 210a toward the ceiling, a distance between the floor and hearing device 210a increases.

When pediatric user 205a utters a sound, hearing device 210a detects voice signal 215a and the echo 216a of voice signal 215a. Because hearing device 210a is farther from the floor, a delay between the time that hearing device 210 detects voice signal 215a and detects the echo 217a is longer. Because the orientation is changed, the attenuation and filtering by the head will also be different. As pediatric user 205a continues to roll, hearing device 210a moves closer to the floor and a delay between a voice signal and an echo of the voice signal will decrease and the attenuation and frequency content of the echo will change. The rotation around the x-axis detected by the inertial measurement unit combined with the changing delay to the echo and changing attenuation and frequency content detected by the acoustic detector of hearing device 210a may indicate that pediatric user 205a is rolling over.

Illustrated in FIG. 2B is a crawling pediatric user 205b and illustrated in FIG. 2C is a walking pediatric user 205c. As illustrated in FIG. 2B, when pediatric user 205b is crawling, an inertial measurement unit of hearing device 210b may detect a movement in the x-direction and an indication that the head of pediatric user 205b is relatively steady, but moving slightly up and down. As illustrated in FIG. 2C, when pediatric user 205c is walking, an inertial measurement unit of hearing device 210c may similarly detect a movement in the x-direction and that the head of pediatric user 205c is relatively steady, but moving slightly up and down. It may be difficult to differentiate between crawling and walking based solely on motion data.

When crawling pediatric user 205b utters a noise or sound, hearing device 210b may detect the voice signal 215b and may additionally detect an echo 216b of the voice signal reverberating off of floor 220b. Similarly, when walking user 205c utters a noise or sound, hearing device 210c may detect the voice signal 215c and may additionally detect an echo 216c of the voice signal reverberating off of floor 220c. Because crawling user 205b is closer to the floor than walking user 205c, a delay between a time when hearing device 210b detects voice signal 215b and detects echo 216b may be shorter than a delay between a time when hearing device 210c detects voice signal 215c and a time when hearing device 210c detects echo 216c. For example, a delay associated with crawling user 205b may be characterized as “short” and a delay associated with walking pediatric user 205c may be delayed as “large.” Based on the length of the delay, a distance of a pediatric user from the floor may be estimated. In addition, the attenuation and filtering relative to the direct sound will also vary with the distance from the floor. A greater distance from the floor or other surface and the presence of the rest of the body of the pediatric user along the return path cause additional attenuation and filtering.

Based on the motion data associated with crawling pediatric user 205b and the short delay between hearing device 210b receiving voice signal 215b and echo 216b, a device, such as external device 110, may determine or predict that pediatric user 205b is crawling. Based on the motion data associated with walking pediatric user 205c and the long delay between hearing device 210c receiving voice signal 215c and echo 216c, the device may determine or predict that pediatric user is walking. The attenuation and filtering relative to the direct signal can also be used to discriminate between crawling and walking.

An estimated pediatric milestone associated with the pediatric user may be logged over time to determine an age at which the pediatric user is reaching the pediatric milestones or to predict when a pediatric user may reach future developmental milestones. For example, based on the motion and microphone data associated with pediatric user 205a, it may be predicted that pediatric user 205a is rolling over, and an indication that pediatric user 205a is rolling over may be logged in a log along with an age (e.g., in months or days) of pediatric user 205a. An age when the pediatric user reaches milestones may be compared to an average age when children reach the milestones to determine whether the pediatric user is on target for reaching the milestones or is reaching the milestones at a delayed rate.

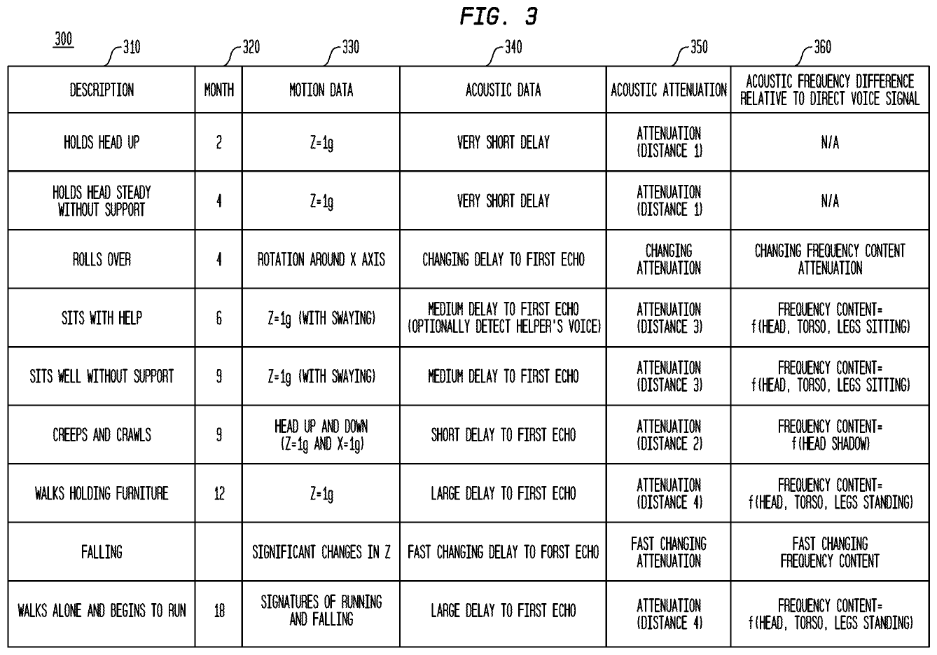

Illustrated in FIG. 3 is an exemplary table 300 with a column 310 indicating a description of pediatric milestones, a column 320 indicating an expected month at which children reach the pediatric milestones, a column 330 indicating motion data that may be detected when a pediatric user is performing an action associated with a pediatric milestone, a column 340 indicating acoustic data that may be associated with the action associated with the pediatric milestone, a column 350 indicating an acoustic attenuation that may be associated with the action associated with the pediatric milestone, and a column 360 indicating an acoustic frequency difference of an echo of a voice of a pediatric user relative to the voice of the pediatric user that may be associated with the action associated with the pediatric milestone.

As illustrated in table 300, a child may be expected to reach the milestone “holds head up” in the child's second month. For a pediatric user, a motion sensor of a hearing device may indicate that Z=1 g when the pediatric user is holding his or her head up. Because the pediatric user is likely close to the floor or another surface, a delay between when a hearing device detects a voice of the pediatric user and a first echo of the voice of the pediatric user may be very short. Additionally, acoustic attenuation data may indicate that the distance between the pediatric user's head and the floor or other surface is small (e.g., distance 1). As further illustrated in table 300, a child may be expected to reach the milestone “holds head steady without support” in the fourth month and the motion sensor of the hearing device may indicate that Z=1 g when a pediatric user reaches this milestone. Similar to when the pediatric user reaches the milestone “holds head up,” the delay between when a hearing device detects a voice of the pediatric user and a first echo of the voice of the pediatric user may be very short and acoustic attenuation data may indicate that the distance between the pediatric user's head and the floor or other surface is small (e.g., distance 1).

As further illustrated in table 300, a child may be expected to reach the milestone “rolls over” in the child's fourth month. As discussed above with respect to FIG. 2A, a hearing device may detect a rotation around the x-axis of a pediatric user and a changing delay to a first echo of the pediatric user's voice when the pediatric user rolls over. Because a location of the hearing device changes while the pediatric user rolls over, the acoustic attenuation and the frequency content change when the child rolls over. As illustrated in table 300, a child may be expected to sit with help in the sixth month. A hearing device of a pediatric user may detect that Z=1 g, possibly with swaying. The hearing device may detect a medium delay to a first echo and the acoustic attenuation may be indicative of a distance of 3, indicating that the hearing device is a medium distance to the floor. Additionally, the hearing device may detect a voice of a person/helper who is supporting the pediatric user. The frequency content of the echo of the voice of the pediatric user may indicate that the echo is being filtered by the head, torso, and sitting legs of the pediatric user.

As further illustrated in table 300, a child may be expected to sit well without support in the ninth month. A hearing device of a pediatric user may detect that Z=1 g and may detect a medium delay to the first echo of the pediatric user's voice and the attenuation may be indicative of a distance of 3, indicating that the hearing device is a medium distance to the floor. Similar to when the pediatric user sits with help, the acoustic frequency data indicates that the echo of the voice is being filtered by the head, torso, and sitting legs of the pediatric user.

As further illustrated in table 300, a child may be expected to creep and crawl by the ninth month. As discussed above with respect to FIG. 2B, a hearing device of a pediatric user may detect that Z=1 g and X=1 g and that the pediatric user's head is moving up and down. In addition the hearing device may detect a short delay to the first echo of the pediatric user's voice and the acoustic attenuation information may indicate a short distance between the hearing device and a floor or other surface (e.g., a distance of 2). The acoustic frequency difference may indicate that the echo is being filtered by a head shadow. A head shadow (or acoustic shadow) is a region of reduced amplitude of a sound because the sound is obstructed by the head. The echo of the voice of the pediatric user may have to travel through and around the head in order to reach an ear. The obstruction caused by the head can account for attenuation (reduced amplitude) of overall intensity as well as cause a filtering effect.

As further illustrated in table 300, a child may be expected to walk holding furniture by the twelfth month. A hearing device of a pediatric user may detect that Z=1 g and, because the pediatric user is standing, the acoustic detector may detect a large delay to the first echo and the acoustic attenuation may indicate a large distance (e.g., a distance of 4) when the pediatric user reaches this milestone. The frequency content may indicate that the echo of the voice of the pediatric user is being filtered by the head, torso, and standing legs of the pediatric user. As further illustrated in table 300, a hearing device of a falling pediatric user may detect significant gravitational changes as well as a fast changing delay to the first echo of the pediatric user's voice and a fast changing attenuation and frequency content of the echo. When a pediatric user begins to walk alone and run, a hearing device of the pediatric user may detect signatures of running and falling. In addition, because the pediatric user is standing, the hearing device may detect a large delay to the first echo. Because the pediatric user is standing, the acoustic attenuation data may indicate a large distance (e.g., a distance of 4) and the frequency content may indicate that the echo is being filtered by the head, torso, and standing legs of the pediatric user. As illustrated in table 300, a child may be expected to reach the milestone of walking alone and beginning to run by the eighteenth month.

The milestones, months, motion data, and acoustic data described above with respect to FIG. 3 are exemplary. Additional milestones associated with a pediatric user may be experienced and logged. In addition, different data may be associated with the milestones described above with respect to FIG. 3.

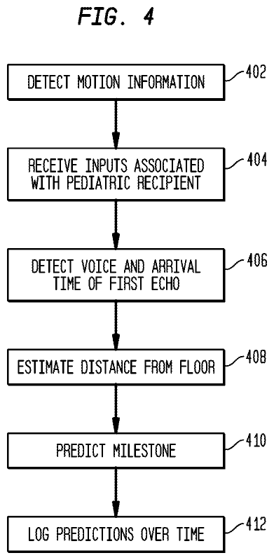

With reference now made to FIG. 4, depicted therein is a flowchart 400 illustrating an example method for implementing the techniques of the present disclosure. The process flow of flowchart 400 begins in operation 402 where motion information associated with a hearing device is determined. For example, sensors included in a hearing device, such as those included in the inertial measurement unit 170 and/or the inertial measurement unit 180 of FIG. 1D, may be used to determine motion information associated with the hearing device.

In operation 404, inputs associated with a pediatric user of a hearing device may be received. For example, a device, such as external device 110 of FIGS. 1A-1D may receive inputs associated with the pediatric user. The inputs may include, for example, a birthdate/age of the pediatric user, a height of the pediatric user, and an indication of whether the pediatric user is experiencing any developmental delay in mobility or is experiencing a balance disorder. Additional and/or different inputs associated with the pediatric user may be received. In some situations, no inputs associated with the pediatric user may be received.

In operation 406, a voice (e.g., a voice of the pediatric user or a voice of a person helping the pediatric user) may be detected. In addition, an arrival time of a first echo of the voice may be determined as well as the frequency content and energy compare to the direct sound of the voice. As discussed above, the first echo of the voice may be received at the hearing device after the voice reverberates off of a floor (or a wall or other surface).

In operation 408, a distance of the hearing device from the floor may be estimated. The distance may be estimated based on a length of the delay between a time when the voice is received at the hearing device and a time when the echo is received at the hearing device. Additionally, the differences in attenuation and frequency content of the direct signal and the echo may be used to determine the distance. If the height of the pediatric user has been received, the height may be used to estimate the distance of the hearing device from the floor or a position of the pediatric user in relation to the floor. In one embodiment, the detection of the echo may be made via autocorrelation. In another embodiment, additional voices may be detected to determine a position of a pediatric user. For example, a detection of a second voice may indicate that another person is helping the pediatric user (e.g., helping the pediatric user to sit up or to walk). A distance of a second voice from the voice of the pediatric user may additionally be used to determine a manner in which a helper is helping a pediatric user. For example, if a time to direct to reverberant ratio is above a threshold it may be determined that the helper is close enough to be in physical contact (e.g., the helper may be helping the pediatric user to sit).

In operation 410, a milestone associated with the pediatric user may be predicted. For example, a device, such as external device 110, may predict the pediatric milestone of the pediatric user using the motion data and the estimated distance of the hearing device from the floor. A current age of the pediatric user and the indication of whether the pediatric user is experiencing a developmental delay in mobility or a balance disorder may be used to interpret or adjust a milestone of the pediatric user. For example, the pediatric milestone may be predicted using table 300 of FIG. 3. In one embodiment, a machine learning algorithm may be used to predict the pediatric milestones based on motion and microphone data associated with a large sample of pediatric users. In this embodiment, the motion and acoustic data in table 300 may be updated based on the machine learning algorithm.

In operation 412, the predicted milestones are logged over time. For example, the device may log an age at which the pediatric user has reached pediatric milestones. The log may be used to determine whether the pediatric user is meeting age appropriate pediatric milestones. Parents or caregivers may be informed whether the pediatric user is tracking to standard milestones. If the pediatric user is not meeting milestones, then additional diagnoses or assistance may be provided. For example, balance/vestibular and general motor problems may be detected by the device and communicated to a clinician or a caregiver. Using the techniques of the present disclosure, balance/vestibular and/or general motor problems may be detected early and rehabilitation may be applied. Furthermore, the techniques may be used to coach family members to provide additional opportunities for movement rehabilitation and the techniques may be used to track whether the interventions are having a desired impact. Additionally, the log of milestones and current motion and acoustic data may be used to predict when a pediatric user is about to reach a new milestone. For example, the device may alert a caregiver/parent when a pediatric user is predicted to walk to for the first time so the caregiver/parent may be there to witness the first steps.

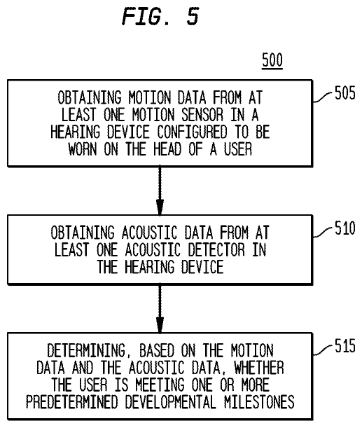

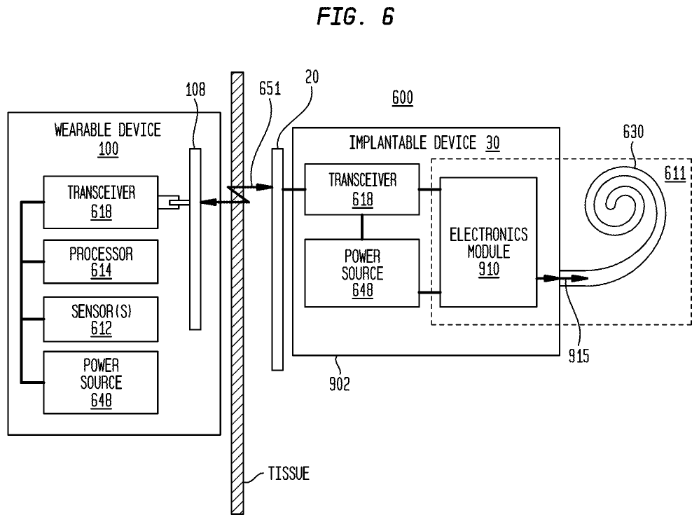

With reference now made to FIG. 5, depicted therein is a flowchart 500 illustrating an example method for implementing the techniques of the present disclosure. The process flow of flowchart 500 begins in operation 505 where motion data is obtained from at least one motion sensor in a hearing device configured to be worn on the head of a user. For example, sensors included in a hearing device, such as those included in the inertial measurement unit 170 and/or the inertial measurement unit 180 of FIG. 1D, may be used to determine motion data associated with the hearing device. The hearing device may be an external or wearable hearing device, or a combination thereof.

In operation 510, acoustic data is obtained from at least one acoustic detector in the hearing device. For example, acoustic data may be obtained from microphones or sound input devices included in the hearing device, such as those included in one or more sound input devices 118 of FIG. 1D. The acoustic data may include information associated with a voice signal of the user and the first echo of the voice signal of the user. For example, the information may include an amount of delay between a time when the acoustic detector receives the voice signal and a time when the acoustic detector receives the echo of the voice signal.

In operation 515, it is determined, based on the motion data and the acoustic data, whether the user is meeting one or more predetermined developmental milestones. For example, the obtained motion data and the acoustic data may be compared to information in table 300 of FIG. 3 to determine whether the user is meeting one or more predetermined developmental milestones.

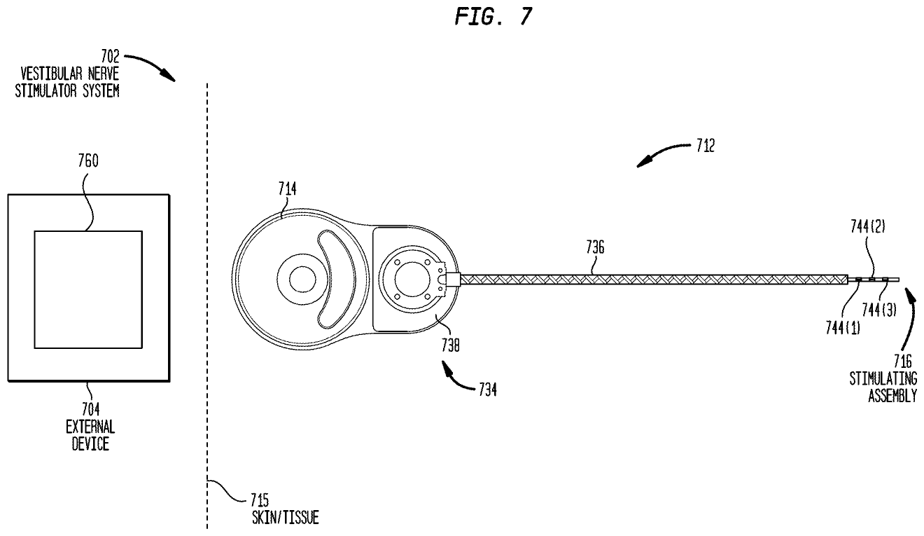

As previously described, the technology disclosed herein can be applied in any of a variety of circumstances and with a variety of different devices. Example devices that can benefit from technology disclosed herein are described in more detail in FIGS. 6 and 7, below. As described below, the operating parameters for the devices described with reference to FIGS. 6 and 7 may be configured according to the techniques described herein. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue, to the extent that the operating parameters of such devices may be tailored based upon the posture of the user receiving the device. Further, technology described herein can also be applied to consumer devices. These different systems and devices can benefit from the technology described herein. For example, the operation techniques of the present disclosure may be applied to consumer grade or commercial grade headphone or ear bud products.

FIG. 6 is a functional block diagram of an implantable stimulator system 600 that can benefit from the technologies described herein. The implantable stimulator system 600 includes the wearable device 100 acting as an external processor device and an implantable device 30 acting as an implanted stimulator device. In examples, the implantable device 30 is an implantable stimulator device configured to be implanted beneath a user's tissue (e.g., skin). In examples, the implantable device 30 includes a biocompatible implantable housing 602. Here, the wearable device 100 is configured to transcutaneously couple with the implantable device 30 via a wireless connection to provide additional functionality to the implantable device 30.

In the illustrated example, the wearable device 100 includes one or more sensors 612, a processor 614, a transceiver 618, and a power source 648. The one or more sensors 612 can be one or more units configured to produce data based on sensed activities. In an example where the stimulation system 600 is an auditory prosthesis system, the one or more sensors 612 include sound input sensors, such as a microphone, an electrical input for an FM hearing system, other components for receiving sound input, or combinations thereof. Where the stimulation system 600 is a visual prosthesis system, the one or more sensors 612 can include one or more cameras or other visual sensors. Where the stimulation system 600 is a cardiac stimulator, the one or more sensors 612 can include cardiac monitors. The processor 614 can be a component (e.g., a central processing unit) configured to control stimulation provided by the implantable device 30. The stimulation can be controlled based on data from the sensor 612, a stimulation schedule, or other data. Where the stimulation system 600 is an auditory prosthesis, the processor 614 can be configured to convert sound signals received from the sensor(s) 612 (e.g., acting as a sound input unit) into signals 651. The transceiver 618 is configured to send the signals 651 in the form of power signals, data signals, combinations thereof (e.g., by interleaving the signals), or other signals. The transceiver 618 can also be configured to receive power or data. Stimulation signals can be generated by the processor 614 and transmitted, using the transceiver 618, to the implantable device 30 for use in providing stimulation.

In the illustrated example, the implantable device 30 includes a transceiver 618, a power source 648, and a medical instrument 611 that includes an electronics module 610 and a stimulator assembly 630. The implantable device 30 further includes a hermetically sealed, biocompatible implantable housing 602 enclosing one or more of the components.

The electronics module 610 can include one or more other components to provide medical device functionality. In many examples, the electronics module 610 includes one or more components for receiving a signal and converting the signal into the stimulation signal 615. The electronics module 610 can further include a stimulator unit. The electronics module 610 can generate or control delivery of the stimulation signals 615 to the stimulator assembly 630. In examples, the electronics module 610 includes one or more processors (e.g., central processing units or microcontrollers) coupled to memory components (e.g., flash memory) storing instructions that when executed cause performance of an operation. In examples, the electronics module 610 generates and monitors parameters associated with generating and delivering the stimulus (e.g., output voltage, output current, or line impedance). In examples, the electronics module 610 generates a telemetry signal (e.g., a data signal) that includes telemetry data. The electronics module 610 can send the telemetry signal to the wearable device 100 or store the telemetry signal in memory for later use or retrieval.

The stimulator assembly 630 can be a component configured to provide stimulation to target tissue. In the illustrated example, the stimulator assembly 630 is an electrode assembly that includes an array of electrode contacts disposed on a lead. The lead can be disposed proximate tissue to be stimulated. Where the system 600 is a cochlear implant system, the stimulator assembly 630 can be inserted into the user's cochlea. The stimulator assembly 630 can be configured to deliver stimulation signals 615 (e.g., electrical stimulation signals) generated by the electronics module 610 to the cochlea to cause the user to experience a hearing percept. In other examples, the stimulator assembly 630 is a vibratory actuator disposed inside or outside of a housing of the implantable device 30 and configured to generate vibrations. The vibratory actuator receives the stimulation signals 615 and, based thereon, generates a mechanical output force in the form of vibrations. The actuator can deliver the vibrations to the skull of the user in a manner that produces motion or vibration of the user's skull, thereby causing a hearing percept by activating the hair cells in the user's cochlea via cochlea fluid motion.

The transceivers 618 can be components configured to transcutaneously receive and/or transmit a signal 651 (e.g., a power signal and/or a data signal). The transceiver 618 can be a collection of one or more components that form part of a transcutaneous energy or data transfer system to transfer the signal 651 between the wearable device 100 and the implantable device 30. Various types of signal transfer, such as electromagnetic, capacitive, and inductive transfer, can be used to usably receive or transmit the signal 651. The transceiver 618 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 648 can be one or more components configured to provide operational power to other components. The power source 648 can be or include one or more rechargeable batteries. Power for the batteries can be received from a source and stored in the battery. The power can then be distributed to the other components as needed for operation.

As should be appreciated, while particular components are described in conjunction with FIG. 6, technology disclosed herein can be applied in any of a variety of circumstances. The above discussion is not meant to suggest that the disclosed techniques are only suitable for implementation within systems akin to that illustrated in and described with respect to FIG. 6. In general, additional configurations can be used to practice the methods and systems herein and/or some aspects described can be excluded without departing from the methods and systems disclosed herein.

FIG. 7 illustrates an example vestibular stimulator system 702, with which embodiments presented herein can be implemented. As shown, the vestibular stimulator system 702 comprises an implantable component (vestibular stimulator) 712 and an external device/component 704 (e.g., external processing device, battery charger, remote control, etc.). The external device 704 comprises a transceiver unit 760. As such, the external device 704 is configured to transfer data (and potentially power) to the vestibular stimulator 712. External device 704 may also include an inertial measurement unit analogous to inertial measurement unit 170 of FIG. 1D.

The vestibular stimulator 712 comprises an implant body (main module) 734, a lead region 736, and a stimulating assembly 716, all configured to be implanted under the skin/tissue (tissue) 715 of the user. The implant body 734 generally comprises a hermetically-sealed housing 738 in which RF interface circuitry, one or more rechargeable batteries, one or more processors, and a stimulator unit are disposed. The implant body 134 also includes an internal/implantable coil 714 that is generally external to the housing 738, but which is connected to the transceiver via a hermetic feedthrough (not shown). Implant body 734 may also include an inertial measurement unit analogous to inertial measurement unit 180 of FIG. 1D.

The stimulating assembly 716 comprises a plurality of electrodes 744(1)-(3) disposed in a carrier member (e.g., a flexible silicone body). In this specific example, the stimulating assembly 716 comprises three (3) stimulation electrodes, referred to as stimulation electrodes 744(1), 744(2), and 744(3). The stimulation electrodes 744(1), 744(2), and 744(3) function as an electrical interface for delivery of electrical stimulation signals to the user's vestibular system.

The stimulating assembly 716 is configured such that a surgeon can implant the stimulating assembly adjacent the user's otolith organs via, for example, the user's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 712, the external device 704, and/or another external device, can be configured to implement the techniques presented herein. That is, the vestibular stimulator 712, possibly in combination with the external device 704 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method comprising:
obtaining motion data from at least one motion sensor in a medical device configured to be worn on a head of a user;
obtaining acoustic data from at least one acoustic detector in the medical device; and
determining, based on the motion data and the acoustic data, whether the user is meeting one or more predetermined developmental milestones.

2. The method of claim 1, further comprising:
determining the user is a pediatric user.

3. The method of claim 1, wherein the one or more predetermined developmental milestones includes one or more of rolling over, sitting, crawling, or walking.

4. The method of claim 1, wherein the motion data is detected by one or more sensors included in the medical device.

5. The method of claim 1, wherein the acoustic data includes an amount of delay between a first time when a voice associated with the user is detected by the at least one acoustic detector and a second time when an echo of the voice is detected by the at least one acoustic detector.

6. The method of claim 5, wherein the acoustic data further includes at least one of attenuation data and frequency data associated with the echo of the voice.

7. The method of claim 1, further comprising:
receiving information associated with the user, wherein the information includes one or more of an age of the user, a height of the user, and an indication of a developmental delay in mobility associated with the user; and
determining whether the user is meeting the one or more predetermined developmental milestones based on the information.

8. The method of claim 1, further comprising:
logging whether the user is meeting the one or more predetermined developmental milestones in a log with the motion data and the acoustic data.

9. One or more non-transitory computer readable storage media comprising instructions that, when executed by a processor, cause the processor to:
obtain motion data from at least one motion sensor in a medical device configured to be worn on a head of a user;
obtain acoustic data from at least one acoustic detector in the medical device; and
determine, based on the motion data and the acoustic data, whether the user is meeting one or more predetermined developmental milestones.

10. (canceled)

11. The one or more non-transitory computer readable storage media of claim 9, wherein the one or more predetermined developmental milestones includes one or more of rolling over, sitting, crawling, or walking.

12. The one or more non-transitory computer readable storage media of claim 9, wherein the motion data is detected by one or more sensors included in the medical device.

13. The one or more non-transitory computer readable storage media of claim 9, wherein the acoustic data includes an amount of delay between a first time when a voice associated with the user is detected by the at least one acoustic detector and a second time when an echo of the voice is detected by the at least one acoustic detector.

14. The one or more non-transitory computer readable storage media of claim 13, wherein the acoustic data further includes attenuation data and frequency data associated with the echo of the voice.

15. The one or more non-transitory computer readable storage media of claim 9, further comprising instructions operable to:
receive information associated with the user, wherein the information includes one or more of an age of the user, a height of the user, and an indication of a developmental delay in mobility associated with the user; and
determine whether the user is meeting the one or more predetermined developmental milestones based on the information.

16. (canceled)

17. A medical device comprising:
one or more motion sensors;
one or more acoustic detectors; and
one or more processors, wherein the one or more processors are configured to:
obtain motion data associated with a user of the medical device from the one or more motion sensors;
obtain acoustic data associated with the user of the medical device from the one or more acoustic detectors; and
determine, based on the motion data and the acoustic data, whether the user of the medical device is meeting one or more predetermined developmental milestones.

18. (canceled)

19. The medical device of claim 17, wherein the one or more predetermined developmental milestones includes one or more of rolling over, sitting, crawling, or walking.

20. The medical device of claim 17, wherein the acoustic data includes an amount of delay between a first time when a voice associated with the user is detected by the one or more acoustic detectors and a second time when an echo of the voice is detected by the one or more acoustic detectors.

21. The medical device of claim 20, wherein the acoustic data further includes one or more of attenuation data and frequency data associated with the echo of the voice.

22. The medical device of 17, wherein the one or more processors are configured to:
receive information associated with the user, wherein the information includes one or more of an age of the user, a height of the user, and an indication of a developmental delay in mobility associated with the user; and
determine whether the user is meeting the one or more predetermined developmental milestones based on the information.

23. (canceled)

24. The method of claim 1, further comprising:
determining, from the acoustic data, an amount of delay between a first time when a voice associated with the user is detected and a second time when an echo of the voice is detected; and
identifying the one or more predetermined developmental milestones based on the motion data and the amount of delay.

25. (canceled)

26. (canceled)

27. (canceled)

28. The method of claim 24, wherein determining the amount of delay includes:
detecting the voice using the at least one acoustic detector included in the medical device; and
detecting the echo of the voice using the the at least one acoustic detector.

29. The method of claim 24, further comprising:
receiving information associated with the user, wherein the information includes one or more of an age of the user, a height of the user, and an indication of a developmental delay in mobility associated with the user; and
identifying the one or more developmental milestones based on the information.

30. (canceled)

31. (canceled)

32. The method of claim 24, wherein determining the amount of delay includes determining the amount of delay via autocorrelation.

33. The method of claim 24, further comprising:
estimating a distance of the user from a floor based on the amount of delay; and
identifying the one or more developmental milestones based on the distance.

34. (canceled)

35. The method of claim 24, further comprising:
predicting a future developmental milestone based on the motion data and the amount of delay.

36. The method of claim 24, wherein identifying the one or more developmental milestones further comprises:
determining attenuation and frequency data associated with the echo of the voice; and
identifying the one or more developmental milestones based on the attenuation and frequency data.
